(19) United States
(12) Patent Application Publication
(10) Pub. No.: US 2020/0222487 A1
(43) Pub. Date: Jul. 16, 2020

(54) ALL-NATURAL TOPICAL PAIN RELIEF OINTMENT

(71) Applicant: Joseph Biernot, Chesapeake, VA (US)

(72) Inventor: Joseph Biernot, Chesapeake, VA (US)

(21) Appl. No.: 16/741,729

(22) Filed: Jan. 13, 2020

Related U.S. Application Data
(60) Provisional application No. 62/791,399, filed on Jan. 11, 2019.

(51) Int. Cl. A61K 36/73; A61K 36/9068; A61K 36/9066; A61K 36/752; A61K 36/88; A61K 36/537; A61K 31/12; A61K 36/71; A61K 36/30; A61K 47/42; A61K 33/06; A61K 36/46; A61K 31/197; A61K 31/198; A61K 31/05; A61K 36/185; A61K 31/10; A61K 31/202; A61K 36/53; A61K 36/534; A61K 36/48; A61K 36/61; A61K 36/74; A61K 36/15; A61K 36/736; A61K 31/015; A61K 31/675; A61K 31/714; A61K 9/06

(52) U.S. Cl. CPC A61K 36/73; A61K 9/06; A61K 36/9066; A61K 36/752; A61K 36/88; A61K 36/537; A61K 31/12; A61K 36/71; A61K 36/30; A61K 47/42; A61K 33/06; A61K 36/46; A61K 31/197; A61K 31/198; A61K 31/05; A61K 36/185; A61K 31/10; A61K 31/202; A61K 36/53; A61K 36/534; A61K 36/48; A61K 36/61; A61K 36/74; A61K 36/15; A61K 36/736; A61K 31/015; A61K 31/675; A61K 31/714; A61K 36/9068

(57) ABSTRACT

The present general inventive concept provides an all-natural pain relief topical ointment composition that is formed from natural ingredients that provides pain relief and other therapeutic benefits for various ailments including arthritis.

### FIELD OF INVENTION

The present general inventive concept relates to a chemical composition, and more particularly, to a pain relief composition including turmeric, grapefruit seed extract, collagen, and various other herbs, extracts and oils which provide pain relief for various ailments, including arthritis.

### BACKGROUND

Millions of people suffer from various physical and skin conditions which cause extreme pain and discomfort. Conventional methods for relieving such pain and discomfort include prescription medications which are composed of synthetic chemical compounds that are often highly addictive and undesirable side effects.

However, there is currently no natural topical ointments that safely and effectively provide pain relief for multiple ailments including arthritis. Therefore, what is desired is a natural topical ointment that provides pain relief for people suffering from arthritis and other ailments without risking adverse side effects.

### SUMMARY OF THE INVENTION

The present general inventive concept provides a pain relief composition for various physical and skin condition including arthritis. The present general inventive concept provides a topical ointment pain relief composition having various natural components that alleviate, reduce, and/or eliminate pain caused by physical and skin conditions and/or diseases. The pain relief composition according to the present general inventive concept comprises alfalfa, apple cider vinegar, apple seed oil, apricot oil, B-5, B-6, B-12, beta carotene, black seed oil, borage seed oil, bromelain, chanca Piedra, chasterberry extract, collagen, Du Zhong, echinachea, eucalyptus, fever few, GABA, GSE, hemp oil, CBD, L-Arginine, lavender, L-Lysine, magnesium, MSM, omega-3, peppermint oil, red clover tea, rosemary oil, sea buckthorn oil, curcumin, tea tree oil, witch hazel, yohimbe, garlic, and various other herb essential oils.

The pain relief composition according to alternative embodiments includes wintergreen oil, camphor oil, menthol oil, and onion and lemon.

The pain relief composition according to the present general inventive concept provides a topical ointment that allows users to treat various types body pain and discomfort caused by arthritis, inflammation, gout, and various other ailments.

The pain relief composition according to the present general inventive concept is a topical ointment that provides therapeutic relief and other benefits for various types of body pain and discomfort caused by arthritis, inflammation, gout, and various other ailments.

The pain relief composition according to the present general inventive concept provides a topical ointment that allows users to treat various types body pain and discomfort caused by arthritis, inflammation, gout, and various other ailments that consists only of natural ingredients.

### DESCRIPTION OF INVENTION

The present general inventive concept provides a topical ointment composition that is formed from natural ingredients that provides pain relief and other therapeutic benefits for various ailments including arthritis. However, the present general inventive concept is not limited thereto.

The topical ointment composition according to the present general inventive concept is configured to provide pain relief and other therapeutic benefits, such as reduction of pain and inflammation for various conditions. The topical ointment composition comprises various ratios of the following compounds, herbs, and extracts including alfalfa, apple cider vinegar, apple seed oil, apricot oil, B-5, B-6, B-12, beta carotene, black seed oil, borage seed oil, bromelain, Chanca Piedra, Chasterberry extract, collagen, Du Zhong, Echinachea, eucalyptus, fever few, GABA, GSE, hemp oil, CBD, L-Arginine, lavender, L-Lysine, magnesium, MSM, omega-3, peppermint oil, red clover tea, witch hazel, yohimbe, garlic, and various other herb essential oils. However, the present general inventive concept is not limited thereto.

In one example embodiment, the topical ointment composition includes a mixture of turmeric, grapefruit seed oil, and one or more of the following components alfalfa, apple cider vinegar, apple seed oil, apricot oil, B-5, B-6, B-12, beta carotene, black seed oil, borage seed oil, bromelain, Chanca Piedra, Chasterberry extract, collagen, Du Zhong, Echinachea, eucalyptus, fever few, GABA, GSE, hemp oil, CBD, L-Arginine, lavender, L-Lysine, magnesium, MSM, omega-3, peppermint oil, red clover tea, rosemary oil, sea buckthorn oil, curcumin, tea tree oil, witch hazel, yohimbe, garlic, and various other herb essential oils. However, the present general inventive concept is not limited thereto.

In exemplary embodiments, one or more of the following compounds or extracts may be distilled and combined in the following percentages:

| Ingredients | Percentage Ranges |
|---|---|
| Alfalfa | 0.5-4% |
| Apple Cider Vinegar | 0.5-8% |
| Apple Seed Oil | 2-10% |
| Apricot Oil | 1-8% |
| B-5 | 1-8% |
| B-6 | 1-8% |
| B-12 | 1-8% |
| Beta Carotene | 0.5-8% |
| Black Seed Oil | 3-12% |
| Borage Seed Oil | 3-12% |
| Bromelain | 2-10% |
| Chanca Piedra | 1-10% |
| Chasteberry Extract | 0.5-4% |
| Collagen | 3-12% |
| Du Zhong | 3-12% |
| Echinachea | 0.5-8% |
| Eucalyptus | 0.5-4% |
| Fever Few | 0.5-4% |
| GABA | 3-12% |
| GSE | 3-12% |
| Hemp Oil/CBD Oil | 0.5-8% |
| L-Arginine | 3-12% |
| Lavender | 3-12% |
| L-Lysine | 3-12% |
| Magnesium | 3-12% |
| MSM* | 0.5-8% |
| Omega-3 | 0.5-8% |
| Peppermint Oil | 0.5-8% |
| Red Clover Tea | 0.5-8% |
| Rosemary Oil | 0.5-8% |
| Sea Buckthorn Oil | 0.5-8% |
| Tea Tree Oil | 0.5-8% |
| Corcumin | 0.5-8% |
| Witch Hazel | 0.5-8% |
| Yohimbe | 0.5-8% |
| Garlic | 0.5-8% |

In another example embodiment, the specific mixture of the above components is configured to provide the maximum amount of therapeutic benefit to the user for various ailments, including arthritis, by including one or more distilled ingredients.

| Ingredients | Percentage Ranges |
|---|---|
| Distilled Ginger Root | 2-15% |
| Distilled Turmeric Root | 2-15% |
| Distilled Grapefruit | 1-8% |
| Distilled Garlic | 1-8% |
| Distilled Chia Seeds | 2.5-14% |
| Casteel Curcumin | 0.2-3% |
| Apple Cider Vinegar | 50-75% |

In alternative example embodiments, the topical ointment composition includes a mixture of turmeric, grapefruit seed oil, and one or more of the following components alfalfa, apple cider vinegar, apple seed oil, apricot oil, B-5, B-6, B-12, beta carotene, black seed oil, borage seed oil, bromelain, Chanca Piedra, Chasterberry extract, collagen, Du Zhong, Echinachea, eucalyptus, fever few, GABA, GSE, pine bark, fo-ti, L-Arginine, lavender, L-Lysine, magnesium, MSM, omega-3, peppermint oil, red clover tea, rosemary oil, sea buckthorn oil, curcumin, tea tree oil, witch hazel, yohimbe, garlic, and various other herb essential oils. However, the present general inventive concept is not limited thereto.

In alternative exemplary embodiments, one or more of the following compounds or extracts may be distilled and combined in the following percentages:

| Ingredients | Percentage Ranges |
|---|---|
| Alfalfa | 0.5-4% |
| Apple Cider Vinegar | 0.5-8% |
| Apple Seed Oil | 2-10% |
| Apricot Oil | 1-8% |
| B-5 | 1-8% |
| B-6 | 1-8% |
| B-12 | 1-8% |
| Beta Carotene | 0.5-8% |
| Black Seed Oil | 3-12% |
| Borage Seed Oil | 3-12% |
| Bromelain | 2-10% |
| Chanca Piedra | 1-10% |
| Chasteberry Extract | 0.5-4% |
| Collagen | 3-12% |
| Du Zhong | 3-12% |
| Echinachea | 0.5-8% |
| Eucalyptus | 0.5-4% |
| Fever Few | 0.5-4% |
| GABA | 3-12% |
| GSE | 3-12% |
| L-Arginine | 3-12% |
| Lavender | 3-12% |
| L-Lysine | 3-12% |
| Magnesium | 3-12% |
| MSM* | 0.5-8% |
| Omega-3 | 0.5-8% |
| Peppermint Oil | 0.5-8% |
| Red Clover Tea | 0.5-8% |
| Rosemary Oil | 0.5-8% |
| Sea Buckthorn Oil | 0.5-8% |
| Tea Tree Oil | 0.5-8% |
| Corcumin | 0.5-8% |
| Witch Hazel | 0.5-8% |
| Yohimbe | 0.5-8% |
| Garlic | 0.5-8% |
| Pine Bark | 0.5-8% |
| Fo-ti | 0.5-8% |

Although a few exemplary embodiments of the present general inventive concept have been illustrated and described, it will be appreciated by those skilled in the art that changes may be made in these exemplary embodiments without departing from the principles and spirit of the general inventive concept, the scope of which is defined in the appended claims and their equivalents.

What is claimed is:

1. A pain relief ointment comprising:
about 50% to about 75% apple cider vinegar;
about 2% to about 15% distilled ginger root; and
about 2% to about 15% distilled turmeric root to make 100%.

2. The pain relief ointment of claim 1, additionally comprising:
about 2.5% to about 14% distilled chia seeds;
about 1% to about 8% distilled grapefruit; and
about 1% to about 8% distilled garlic.

3. The pain relief ointment of claim 2, additionally comprising:
about 0.2% to about 3% casteel curcumin.

4. A pain relief ointment comprising:
about 3% to about 12% black seed oil;
about 3% to about 12% borage seed oil;
about 3% to about 12% collagen; and
about 3% to about 12% Magnesium to make 100%.

5. The pain relief ointment of claim 4, additionally comprising:
about 3% to about 12% Du Zhong;
about 3% to about 12% GABA;
about 3% to about 12% L-Arginine;
about 3% to about 12% Lavender; and
about 3% to about 12% L-Lysine.

6. The pain relief ointment of claim 5, additionally comprising:
about 3% to about 12% Du Zhong.

7. The pain relief ointment of claim 5, additionally comprising:
about 0.5% to about 8% Hemp Oil.

8. The pain relief ointment of claim 5, additionally comprising:
about 0.5% to about 8% CBD Oil.

9. The pain relief ointment of claim 6, additionally comprising:
about 0.5% to about 8% MSM.

10. The pain relief ointment of claim 6, additionally comprising:
about 0.5% to about 8% Omega-3;
about 0.5% to about 8% peppermint oil;
about 0.5% to about 8% red clover tea;
about 0.5% to about 8% rosemary oil;
about 0.5% to about 8% sea buckthorn oil; and
about 0.5% to about 8% tea tree oil.

11. The pain relief ointment of claim 10, additionally comprising:
about 0.5% to about 8% corcumin;
about 0.5% to about 8% witch hazel;
about 0.5% to about 8% yohimbe; and
about 0.5% to about 8% garlic.

12. A pain relief ointment comprising:
about 0.5% to about 8% distilled apple cider vinegar;
about 3% to about 12% GABA;
about 3% to about 12% GSE; and
about 3% to about 12% Magnesium to make 100%.

13. The pain relief ointment of claim 12, additionally comprising:
about 0.5% to about 8% pine bark;
about 0.5% to about 8% Fo-ti;
about 3% to about 12% L-Arginine;
about 3% to about 12% Lavender; and
about 3% to about 12% L-Lysine.

14. The pain relief ointment of claim 13, additionally comprising:
about 3% to about 12% Black Seed Oil; and
about 3% to about 12% Borage See Oil.

15. The pain relief ointment of claim 13, additionally comprising:
about 0.5% to about 8% Hemp Oil.

16. The pain relief ointment of claim 13, additionally comprising:
about 0.5% to about 8% CBD Oil.

17. The pain relief ointment of claim 14, additionally comprising:
about 3% to about 10% bromelain;
about 1% to about 10% chanca piedra;
about 3% to about 12% collagen; and
about 3% to about 12% Du Zhong.

18. The pain relief ointment of claim 17, additionally comprising:
about 0.5% to about 8% corcumin;
about 0.5% to about 8% witch hazel;
about 0.5% to about 8% MSM;
about 0.5% to about 8% Omega-3;
about 0.5% to about 8% peppermint oil;
about 0.5% to about 8% red clover tea;
about 0.5% to about 8% rosemary oil;
about 0.5% to about 8% sea buckthorn oil;
about 0.5% to about 8% tea tree oil;
about 0.5% to about 4% alfalfa;
about 2% to about 10% apple seed oil;
about 1% to about 8% apricot oil;
about 1% to about 8% B-5;
about 1% to about 8% B-6;
about 1% to about 8% B-12;
about 0.5% to about 8% garlic;
about 0.5% to about 8% yohimbe; and
about 0.5% to about 8% beta carotene.
